(19) United States
(12) Patent Application Publication
(10) Pub. No.: US 2018/0132734 A1
(43) Pub. Date: May 17, 2018

(54) ACTIVE BIOMONITOR

(71) Applicant: Jose Carmelo Martinez, Toms River, NJ (US)

(72) Inventor: Jose Carmelo Martinez, Toms River, NJ (US)

(21) Appl. No.: 15/353,125

(22) Filed: Nov. 16, 2016

(51) Int. Cl. A61B 5/0205; A61B 5/00; A61B 5/024

(52) U.S. Cl. CPC A61B 5/02055; A61B 5/746; A61B 5/6824; A61B 5/0022; A61B 5/024

(57) ABSTRACT

A biomonitor having various functionality for actively monitoring certain vital signs and wellness data is provided. The biomonitor is adapted to compare the wellness data to programmed and/or determined thresholds and call 911 if such wellness data indicates a medical emergency.

### BACKGROUND OF THE INVENTION

The present invention relates to health monitoring devices and, more particularly, to a biomonitor providing various functionality for actively monitoring certain vital signs and wellness data.

Sudden Infant Death Syndrome (SIDS) is the leading cause of death in infant newborns 12-months of age. In 2010 over two thousand infants passed away from SIDS. On the average a child who has died from SIDS will show signs of aspirating or choking. Accidental suffocations, claims another 665 infants per year. Aside from this you have infants and children with asthma, epilepsy, diabetes and other congenital diseases that need constant monitoring but cannot be admitted to a hospital. Add to this the fact that many parents cannot afford a stay at home nurse 24/7. Many infants and child congenital diseases manifest as a compromised airway, causing a dramatic change in the child's heart rate and oxygen level (cardiac arrest is usually a result of respiratory arrest in a child).

Current monitors only passively monitor vital signs, but are unable to actively assist in responding to a medical emergency the vital signs indicate.

As can be seen, there is a need for an improved biomonitor for children so as to actively monitoring certain vital signs.

### SUMMARY OF THE INVENTION

In one aspect of the present invention, a biomonitor providing various functionality for actively monitoring certain vital signs and wellness data includes a body housing a plurality of wellness sensors configured for measuring a plurality of vital signs of a human user; a band connected to the body, wherein the band is adapted for securing the body to the human user so that the plurality of wellness sensors operably engages the human user; a microprocessor coupled to the plurality of wellness sensors, wherein the microprocessor is configured to categorize the plurality of vital signs in one of four categories including critical, unstable, potential, and stable; and a software application loaded on the microprocessor, wherein the software application is configured to automatically call 911 if the plurality of vital signs is categorized as critical or unstable.

In another aspect of the present invention, the biomonitor providing various functionality for actively monitoring certain vital signs and wellness data includes a body housing a plurality of wellness sensors configured for measuring a plurality of vital signs of a human user; the plurality of wellness sensors includes a tri-sensor pulse oximetry sensor, at least one temperature sensor, and a microphone sensor; a band connected to the body, wherein the band is adapted for securing the body to the human user so that the plurality of wellness sensors operably engages the human user; a microprocessor coupled to the plurality of wellness sensors, wherein the microprocessor is configured to categorize the plurality of vital signs in one of four categories including critical, unstable, potential, and stable; a speaker coupled to the microprocessor; a display screen coupled to the microprocessor; a LED light coupled to the microprocessor; a GPS coupled to the microprocessor, wherein the GPS is configured to define a physical location of the device; a software application loaded on the microprocessor, wherein the software application is configured to automatically call 911 if the plurality of vital signs is categorized as critical or unstable through a three-tone prompt transmitting the physical location of the device, the plurality of vital signs of the human user, and the categorical identification of the plurality of vital signs; and the microprocessor configured to: output through the speaker and or the display screen at least one recorded instruction tailored to at least one medical need of the human user; and activate the LED light when automatically calling 911.

These and other features, aspects and advantages of the present invention will become better understood with reference to the following drawings, description and claims.

### BRIEF DESCRIPTION OF THE DRAWINGS

FIG. 1 is a schematic view of an exemplary embodiment of the present invention;

FIG. 2 is a perspective view of an exemplary embodiment of the present invention; and

FIG. 3 is a flowchart of an exemplary embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a biomonitor providing various functionality for actively monitoring certain vital signs and wellness data, wherein the biomonitor is adapted to compare the wellness data to programmed and/or determined thresholds and call 911 if such wellness data indicates a medical emergency.

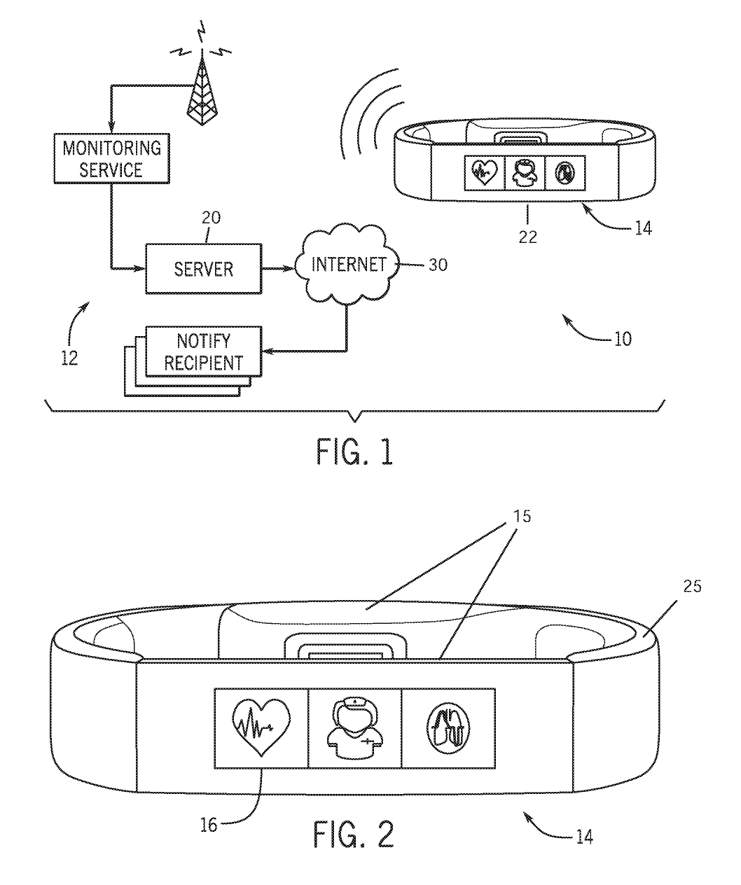

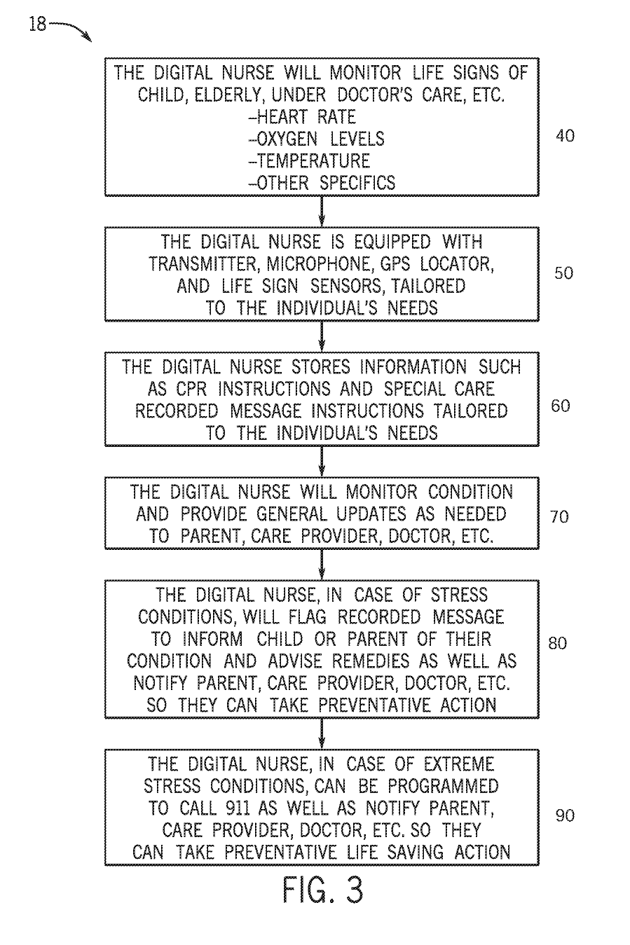

Referring to FIG. 1, the present invention may include at least one computer with a user interface. The computer may include at least one processing unit coupled to a form of memory including, but not limited to, a computing device, a server 20, a microprocessor, a desktop, a laptop, and smart device, such as a tablet, a smart phone, smart watch, a smart bracelet 22, or the like. The computer may include a program product including a machine-readable program code for causing, when executed, the computer to perform steps. The program product may include software which may either be loaded onto the computer or accessed by the computer. The loaded software may include an application on a smart device. The software may be accessed by the computer using a web browser. The computer may access the software via the web browser using the internet 30, extranet, intranet, host server, internet cloud, wifi network, and the like.

Referring to FIG. 2, the present invention may include a biomonitor 10 for detecting life threats and actively alerting assistance to said life threats. The present invention may be called the “Digital Nurse”. In certain embodiments, the biomonitor 10 may be designed to detect life threats to children with congenital diseases and to automatically call for the appropriate resources to help save the child. The biomonitor 10 may provide a body (or bodies) 15 connected to a wrist band 25 dimensioned and adapted to secure the biomonitor 10 around the wrist of a user. By constantly monitoring vital signs, such as their temperature, heart rate, blood pressure, respiratory rate and pulse and oxygen level, and whether there is a marked or critical change in said vital signs, the biomonitor 10 is able to react to a medical emergency of the user. If the vitals reach a dangerous or critical level, then the biomonitor 10 is adapted to take the appropriate action to alert the necessary resources, as well as be adapted to provide give onsite CPR instruction to those on the scene. Upon detecting a child in cardiac or respiratory arrest, for example, the biomonitor 10 with its “Auto 911” feature calls for an ambulance, such as by calling 911, and through the call provides the child's name, present location, condition and last set of vital signs taken.

The present invention may include the following primary components 14: a microprocessor, such as the MCP2515, with sufficient memory and firmware and a software application, Bluetooth functionality, GPS functionality, an output signal, such as an LED light, a power source, a user interface 16, a plurality of wellness sensors including a tri-sensor pulse oximetry sensor; at least one temperature sensor; and a microphone sensor, as well as other wellness sensors adapted for the following: in cancer patients the detection of new cancer cells or the remission of existing ones for better monitoring and follow up treatment; detection of central nervous system or synapse irregularities to treat or prevent seizures; detection of rising vascular pressure to treat or prevent Cerebral Vascular Accidents or Transient Ischemic Attacks (Stroke patients); detection of rising Systolic/Diastolic pressure to treat or prevent Myocardial Infarctions (Heart Attacks); and detection of rising or dropping glycogen or insulin levels for the treatment of diabetics.

The above-mentioned wellness sensors, functionality, power source and output signal may be coupled to the microprocessor so as to be controlled by the software application for measuring wellness data. These primary components 14 may be encased in the body 15 of the biomonitor 10. The biomonitor 10 may be made of a hyper allergenic waterproof material and may come in a variety of colors and designs. The bracelet may be flexible and comfortable to accommodate a child's movement. Along the body 15, the wellness sensors may be disposed for interfacing with skin of the wearer-user.

The software application may be adapted to provide the following features: Multi-language capability (using Google® translate); Auto GPS (using Google® Maps); Auto Temperature read; Auto Vital Signs read; Auto CPR instruction based on AMA and AHA standards; Auto C.U.P.S. status—based on the numerical values of the vital signs the app may place the child in one of four categories (Critical, Unstable, Potential Unstable, Stable); and Auto 911 with message.

The biomonitor 10 enables a parent to monitor the heart rate, oxygen level and temperature in a child and alerts the parent in case there is an emergent change in these readings. Each may include the wellness sensors that may continuously monitor the heart rate, oxygen level and temperature in both infants and children. The readings are displayed in number values that is easy for any parent to read and understand. The unit may also include a built-in microphone so that the parent can hear the child. The biomonitor 10 may also work with the software application that may be compatible with smart devices, including but not limited to smart phones, laptops, desktops, tablets and smart TV's. Each unit can be personalized to a child's name, date of birth, and weight. For example, through user interface 16, a user can navigate the onscreen instructions to fill in the user's personal information, medical condition, related medications and allergies, via prompts, multiple choice answers, or the like. Based on this information the unit may then calibrate to the child's proper heart rate and oxygen level.

The microprocessor may provide an alarm system 12 adapted to analyze the wellness data so that, in one embodiment, the microprocessor generates alarms if the value of any of the wellness data exceeds predetermined thresholds, wherein said thresholds are likely established by medical professionals. In such a case, microprocessor may generate exportable messages, for example phone messages, email messages and SMS messages sent to user-specified addresses, such as 911. Biomonitor 10 may be adapted to activate the output signal. The alarm system may be coupled with the GPS, WiFi, and GTS so as the above-mentioned alarm message (911 call, SMS or email) includes the user's current location even if the child is unconscious.

If the biomonitor 10 detects a Critical or Unstable level in the child's vital signs 911 is automatically called. A three-tone prompt may let the 911 receiving operator know that the call is from the biomonitor 10. The message may include the child's name, present location and the status of the child (Cardiac Arrest or Respiratory Arrest) and last recorded set of vital signs. The message may repeat itself until the 911 operator disengages the call. Auto 911 messages may be delivered on in English or other languages. The output signal may blink in RED and White continuously when Auto 911 is activated. This may also aid in the recovery of a child in the event that the child is lost in a low-lit area or in a mass crowd. Through the Auto CPR feature, the biomonitor 10 may also be able to give directions on either Rescue Breathing or CPR as per the American Heart Association and American Medical Association Guidelines. This can be done by hitting the Auto CPR icon on the phone or by pressing the biomonitor 10 face on the monitor.

The above features can be activated by the parent by checking off the appropriate boxes. The components may detect critical changes in heart, stroke, diabetic, cancer, and epileptic patients. The configuration can also be applied to patients who are already admitted to a hospital, hospice or nursing home for total and complete monitoring of the patient.

A method of using the present invention may include the following. The biomonitor 10 disclosed above may be provided. A user may secure the biomonitor 10 about the wrist of a child after programing the microprocessor to set the thresholds associated with each wellness sensor.

Additionally, for the elderly the biomonitor 10 may erase the fear of living alone by providing the confidence that if the user's medical condition should for whatever reason put them in a critical situation that the biomonitor 10 is not just there to monitor but to make that much needed 911 call to get them the help they desperately need. Better than a medical alert bracelet or any system that requires activation and verbal response the Digital Nurse will have all the necessary information stored and ready for all necessary resources that need it. This is especially critical if the elderly patient should fall unconscious due to illness or injury. In the nursing home setting where there is limited staffing and so many residents the biomonitor 10 is indispensable. Its constant monitoring of the patient will give family members peace of mind as they are able to see their loved one's present physical state.

In a hospital setting the biomonitor 10 applications can be numerous. First it acts as a digital ID band for each patient. Second its monitoring system can be implemented into the facilities computer network giving doctors immediate on sight data of each patient. Third nurses can leave the mundane task of taking vital signs and temperatures. The biomonitor 10 with its constant monitoring of these vital systems will free nurses to do more important patient related tasks and at the same time be more readily available to assist doctors in more serious medical issues. This is especially ideal for a facility with limited medical staffing or if tending to a major catastrophe or event. Lastly for those patients who are highly contagious and who have to be separated or quarantined the biomonitor 10 will reduce the risk of exposure to hospital workers for reasons of vital signs and temperature taking.

Also, the biomonitor 10 can be implemented by both the Center for Disease Control and The World Health Organization in areas where there might be an outbreak of Ebola, Swine Flu, Malaria, Leprosy and Avian Influenza. By limiting contact between the health care workers and those infected the spread of infection is thereby limited. All this while still maintaining a constant watch of every patients vital functions and temperature. On a large scale like this not a single patient will be lost in the shuffle. The biomonitor 10 can do the job of monitoring these patients while giving the medical staff the freedom to treat the more serious, once again minimizing cost of staffing and still saving lives.

In method 18, beginning with step 40, the digital nurse (biomonitor 10) may monitor life signs of the wearer (child, elderly, anyone under a medical professional's care), including their heart rate, oxygen levels, temperature, and other specifics. In step 50, the biomonitor 10 may be equipped with a transmitter, microphone, GPS locator, and life sign/wellness sensors tailored to the user's medical needs. In step 60, the biomonitor 10 may store information such as CPR instructions and special care recorded message instructions tailored to the user's medical needs. In step 70, the biomonitor 10 may monitor wellness data/conditions of the user, providing general updates as needed to parents, care providers, medical professionals and the like. In step 80, the biomonitor 10 in the case of stress conditions may flag recorded message to inform child or parent of their condition and advise remedies as well as notify parents, care provider, medical professionals and the like so they can take preventative action. In step 90, the biomonitor 10 in case of extreme stress conditions can be programmed to call 911 as well as notify parents, care provider, medical professionals and the like so they can take preventative life saving action.

The computer-based data processing system and method described above is for purposes of example only, and may be implemented in any type of computer system or programming or processing environment, or in a computer program, alone or in conjunction with hardware. The present invention may also be implemented in software stored on a computer-readable medium and executed as a computer program on a general purpose or special purpose computer. For clarity, only those aspects of the system germane to the invention are described, and product details well known in the art are omitted. For the same reason, the computer hardware is not described in further detail. It should thus be understood that the invention is not limited to any specific computer language, program, or computer. It is further contemplated that the present invention may be run on a stand-alone computer system, or may be run from a server computer system that can be accessed by a plurality of client computer systems interconnected over an intranet network, or that is accessible to clients over the Internet. In addition, many embodiments of the present invention have application to a wide range of industries. To the extent the present application discloses a system, the method implemented by that system, as well as software stored on a computer-readable medium and executed as a computer program to perform the method on a general purpose or special purpose computer, are within the scope of the present invention. Further, to the extent the present application discloses a method, a system of apparatuses configured to implement the method are within the scope of the present invention.

It should be understood, of course, that the foregoing relates to exemplary embodiments of the invention and that modifications may be made without departing from the spirit and scope of the invention as set forth in the following claims.

What is claimed is:

1. A device, comprising:
a body housing a plurality of wellness sensors configured for measuring a plurality of vital signs of a human user;
a band connected to the body, wherein the band is adapted for securing the body to the human user so that the plurality of wellness sensors operably engages the human user;
a microprocessor coupled to the plurality of wellness sensors, wherein the microprocessor is configured to categorize the plurality of vital signs in one of four categories including critical, unstable, potential, and stable; and
a software application loaded on the microprocessor, wherein the software application is configured to automatically call 911 if the plurality of vital signs is categorized as critical or unstable.

2. The device of claim 1, further comprising a speaker coupled to the microprocessor, wherein the microprocessor is configured to output through the speaker at least one recorded instruction tailored to at least one medical need of the human user.

3. The device of claim 2, wherein the at least one recorded instruction includes CPR instructions.

4. The device of claim 1, further comprising a display screen coupled to the microprocessor, wherein the microprocessor is configured to output through the display screen at least one recorded instruction tailored to at least one medical need of the human user.

5. The device of claim 1, wherein the plurality of wellness sensors further includes a tri-sensor pulse oximetry sensor, at least one temperature sensor, and a microphone sensor.

6. The device of claim 1, further including a LED light coupled to the microprocessor, wherein the microprocessor is configured to activate the LED light when automatically calling 911.

7. The device of claim 1, further including a GPS coupled to the microprocessor, wherein the GPS is configured to define a physical location of the device.

8. The device of claim 7, wherein the software application is further configured to provide 911 a three-tone prompt transmitting the physical location of the device, the plurality of vital signs of the human user, and the categorical identification of the plurality of vital signs.

9. A device, comprising:
a body housing a plurality of wellness sensors configured for measuring a plurality of vital signs of a human user;
the plurality of wellness sensors comprising a tri-sensor pulse oximetry sensor, at least one temperature sensor, and a microphone sensor;
a band connected to the body, wherein the band is adapted for securing the body to the human user so that the plurality of wellness sensors operably engages the human user;
a microprocessor coupled to the plurality of wellness sensors, wherein the microprocessor is configured to categorize the plurality of vital signs in one of four categories including critical, unstable, potential, and stable;
a speaker coupled to the microprocessor;
a display screen coupled to the microprocessor;
a LED light coupled to the microprocessor;
a GPS coupled to the microprocessor, wherein the GPS is configured to define a physical location of the device;
a software application loaded on the microprocessor, wherein the software application is configured to automatically call 911 if the plurality of vital signs is categorized as critical or unstable through a three-tone prompt transmitting the physical location of the device, the plurality of vital signs of the human user, and the categorical identification of the plurality of vital signs; and
the microprocessor configured to:
output through the speaker and or the display screen at least one recorded instruction tailored to at least one medical need of the human user; and
activate the LED light when automatically calling 911.

10. The device of claim 9, wherein the at least one recorded instruction includes CPR instructions.
